(19) United States
(12) Patent Application Publication
(10) Pub. No.: US 2024/0092713 A1
(43) Pub. Date: Mar. 21, 2024

(54) ION-GATED NANOCHANNEL CATALYTIC MEMBRANE REACTOR AND METHODS/PROCESS FOR CHEMICALS AND FUELS PRODUCTION

(71) Applicant: E2H2NANO, LLC., Buffalo, NY (US)

(72) Inventor: Shoujie Ren, Buffalo, NY (US)

(73) Assignee: E2H2NANO, LLC., Buffalo, NY (US)

(21) Appl. No.: 18/370,643

(22) Filed: Sep. 20, 2023

Related U.S. Application Data
(60) Provisional application No. 63/408,613, filed on Sep. 21, 2022.

(51) Int. Cl. C07C 29/152; C01C 1/04; C07C 29/151; C07C 29/153; C07C 29/74; C07C 68/00

(52) U.S. Cl. CPC C07C 29/152; C01C 1/0411; C01C 1/0417; C01C 1/0458; C07C 29/1512; C07C 29/153; C07C 29/74; C07C 68/00

(57) ABSTRACT

Various aspects of an ion-gated nanochannel catalytic membrane reactor, disclosed herein. The ion-gated nanochannel catalytic membrane reactor includes a reaction unit comprising at least a plurality of reactants that react to produce one or more permeates at least due to activation by a catalyst. The membrane reactor further includes a separation unit including an ion-gated nanochannel membrane supported on a hollow fiber support. The polar permeates from the one or more permeates pass through the ion-gated nanochannel membrane and are separated in-situ. Multiple methods/processes using the ion-gated nanochannel catalytic membrane reactor for chemicals and fuels production are included.

### CROSS REFERENCE TO RELATED APPLICATIONS

This patent application claims the benefit of U.S. Prov. Pat. App. Ser. No. 63/408,613 filed Sep. 21, 2022, which is incorporated herein.

### STATEMENT REGARDING FEDERALLY SPONSORED RESEARCH AND DEVELOPMENT

This invention was made with government support under award No. DE-EE0009409 awarded by the Department of Energy. The government has certain rights in the invention.

### FIELD OF THE DISCLOSURE

The present disclosure generally relates to the field of membrane reactor, and in particular, the present disclosure relates to method and system for providing an ion-gated nanochannel catalytic membrane reactor and methods/processes for chemicals and fuels production.

### BACKGROUND OF THE DISCLOSURE

Many processes have been developed to improve the conversion and product yield for the thermodynamically limited reactions for chemical and fuel production. These processes typically use reactive distillation, vacuum, or sorption to remove one of products in order to overcome thermodynamic limitations and achieve high conversion rate and product yield.

However, the reactive distillation process and vacuum is energy intensive. Sorption is required to regenerate the absorbents for reuse. These processes also have a limitation for some reactions which involve the production of polar molecules, such as water. For example, water could form an azeotropic mixture with other products or reactant that makes the distillation very difficult. The membrane technique has been developed for the separation of gas or liquid in many fields.

Membrane reactor with combined reaction and separation in one unit has attracted much attention. Water is a byproduct in many catalytic reactions, and its removal is important in improving the production rate or the separation with the products. Membrane reactors employ different membrane materials, including ZSM-5, PVA, H-ultra-stable Y (USY), NaA zeolite, etc., have been reported for water removal. However, the stability of membrane and the reactor at high pressures (>10 bar) and high temperatures (>200° C.) is challenged. In addition to reactions involving the water formation, there are some reactions involving other polar compounds, such as NH3. These reactions are also thermodynamically limited. However, no membrane reactors are reported for these reactions.

In light of the above listed problems, there is a need for a system and method for production of compounds that leads to enhancement of conversion and yield in production of compounds.

Further, there is a need for a system and method that eliminated distillation or cryogenic condensation and reheating of unreacted reactants when recycling. Furthermore, there is a need for a system and method that improves lifetime of a catalyst and leads to reduction in cost for production of compounds.

### SUMMARY OF THE DISCLOSURE

The foregoing summary is illustrative only and is not intended to be in any way limiting. In addition to the illustrative aspects, embodiments, and features described above, further aspects, embodiments, and features will become apparent by reference to the drawings and the following detailed description.

An ion-gated nanochannel catalytic membrane reactor as shown in, and/or described in connection with, at least one of the figures, as set forth more completely in the description below. The ion-gated nanochannel catalytic membrane reactor includes a reaction unit comprising at least a plurality of reactants that react to produce one or more permeates at least due to activation by a catalyst. The membrane reactor further includes a separation unit including an ion-gated nanochannel membrane supported on a hollow fiber support. The polar permeates from the one or more permeates pass through the ion-gated nanochannel membrane and are separated in-situ.

A method for production of compounds using an ion-gated nanochannel catalytic membrane reactor as shown in, and/or described in connection with, at least one of the figures, as set forth more completely in the description below. The method includes providing, in a reaction unit, at least a plurality of reactants that react to produce one or more permeates at least due to activation by a catalyst. The method further includes separating in-situ, from a separation unit, polar permeates from the one or more permeates that pass through the ion-gated nanochannel membrane of the reactor. The separation unit comprises an ion-gated nanochannel membrane supported on a hollow fiber support.

These and other features and advantages of the present disclosure may be appreciated from a review of the following detailed description of the present disclosure, along with the accompanying figures in which like reference numerals refer to like parts throughout.

### BRIEF DESCRIPTION OF DRAWINGS

This disclosure will be more fully understood from the following detailed description taken in conjunction with the accompanying drawings, in which:

FIG. 1 depicts a schematic diagram of ion-gated nanochannel catalytic membrane reactor 100, in accordance with an embodiment of the invention;

FIG. 2 shows a simplified schematic view 200 of reaction unit 102 and the separation unit 104, in accordance with an embodiment of the invention;

With reference to FIG. 3, there is shown the reactor in which the catalyst activates the plurality of reactants at the desired temperature and pressure, in accordance with an embodiment of the invention;

FIG. 4A is a schematic view that depicts the presence of metal ions in the channel of the ion-gated nanochannel membrane and the permeation of polar small molecules through the ion-gated nanochannel membrane, in accordance with an embodiment of the invention; and

FIG. 4B depicts separation ammonia (NH3) using the ion-gated nanochannel membrane during ammonia synthesis, in accordance with an embodiment of the invention.

FIG. 5 depicts a flow diagram of a method for production of compounds using an ion-gated nanochannel catalytic membrane reactor, in accordance with some embodiments of the present disclosure.

### DETAILED DESCRIPTION OF THE DISCLOSURE

The present disclosure will now be described more fully with reference to the accompanying drawings, in which embodiments of the present disclosure are shown. However, this disclosure should not be construed as limited to the embodiments set forth herein. Rather, these embodiments are provided so that this disclosure will be thorough and complete, and will fully convey the scope of the present disclosure to those skilled in the art. In the present disclosure, like-numbered components of various embodiments generally have similar features when those components are of a similar nature and/or serve a similar purpose.

The terms “a” and “an” herein do not denote a limitation of quantity, but rather denote the presence of at least one of the referenced item.

The term “having”, “including” and “comprising” herein refer to inclusion of at least one of the referenced items.

Various implementations may be found in ion-gated nanochannel catalytic membrane reactor, is disclosed. The ion-gated nanochannel catalytic membrane reactor includes a reaction unit including at least a plurality of reactants that react to produce one or more permeates at least due to activation by a catalyst. The membrane reactor further includes a separation unit including an ion-gated nanochannel membrane supported on a hollow fiber support. The polar permeates from the one or more permeates pass through the ion-gated nanochannel membrane and are separated in-situ.

In accordance with an embodiment, the ion-gated nanochannel membrane may be coated on a ceramic hollow fiber support. This enables the operation of the membrane reactor at high temperature (>300° C.) and pressure (>30 bar).

In accordance with an embodiment, the ion-gated nanochannel membrane comprises metal ions that are one of Lithium (Li), Sodium (Na), Potassium (K), Rubidium (Rb), or Cesium (Cs). The metal ions are incorporated in the ion-gated nanochannel membrane by an ion exchange mechanism with sodium zeolite membrane. The passing through of the polar permeates, may be based on manipulation of the size of a plurality of pores of the ion-gated nanochannel membrane by selecting the metal ions in accordance with the size of the polar permeates.

In accordance with an embodiment, using the ion-gated nanochannel catalytic membrane reactor, production of methanol (CH3OH) may be performed by providing, in a reaction unit, carbon dioxide (CO2) and hydrogen (H2) as reactants for producing methanol (CH3OH) and polar water (H2O) as permeates due to activation by a catalyst, an ambient temperature (150° C.-300° C.), and pressure of (10-70 bar). Subsequently, from a separation unit, the polar water (H2O) produced as permeate may be separated in-situ as it passes through the ion-gated nanochannel membrane of the reactor.

In accordance with an embodiment, using the ion-gated nanochannel catalytic membrane reactor, production of ethanol (CH3CH2OH) may be performed by providing, in a reaction unit, carbon dioxide (CO2) and hydrogen (H2) as reactants for producing ethanol (CH3CH2OH) and polar water (H2O) as permeates due to activation by a catalyst, an ambient temperature (150° C.-400° C.), and pressure of (10-70 bar). Subsequently, from a separation unit, the polar water (H2O) produced as permeate may be separated in-situ as it passes through the ion-gated nanochannel membrane of the reactor.

In accordance with an embodiment, using the ion-gated nanochannel catalytic membrane reactor, production of olefins (CnH2n) may be performed by providing, in a reaction unit, carbon dioxide (CO2) and hydrogen (H2) as reactants for producing olefins (CnH2n) and polar water (H2O) as permeates due to activation by a catalyst, an ambient temperature (150° C.-400° C.), and pressure of (10-70 bar). Subsequently, from a separation unit, the polar water (H2O) produced as permeate may be separated in-situ as it passes through the ion-gated nanochannel membrane of the reactor.

In accordance with an embodiment, using the ion-gated nanochannel catalytic membrane reactor, production of gasoline (C6-C10) and jet fuel range (C8-C15) hydrocarbonsmay be performed by providing, in a reaction unit, carbon dioxide (CO2) and hydrogen (H2) as reactants for producing gasoline (C6-C10) and jet fuel range (C8-C15) hydrocarbons and polar water (H2O) as permeates due to activation by a catalyst, an ambient temperature (150° C.-400° C.), and pressure of (10-70 bar). Subsequently, from a separation unit, the polar water (H2O) produced as permeate may be separated in-situ as it passes through the ion-gated nanochannel membrane of the reactor.

In accordance with an embodiment, using the ion-gated nanochannel catalytic membrane reactor, production of methane (CH4) may be performed by providing, in a reaction unit, carbon dioxide (CO2) and hydrogen (H2) as reactants for producing methane (CH4) and polar water (H2O) as permeates due to activation by a catalyst, an ambient temperature (150° C.-400° C.), and pressure of (10-70 bar). Subsequently, from a separation unit, the polar water (H2O) produced as permeate may be separated in-situ as it passes through the ion-gated nanochannel membrane of the reactor.

In accordance with an embodiment, using the ion-gated nanochannel catalytic membrane reactor, production of ammonia (NH3) may be performed using the ion-gated nanochannel catalytic membrane reactor by providing, in a reaction unit, nitrogen (N2) and hydrogen (H2) as reactants for producing ammonia (NH3) as permeate due to activation by a catalyst, an ambient temperature (200° C.-400° C.), and pressure of (10-70 bar). Subsequently, from a separation unit, the ammonia (NH3) produced as permeate may be separated in-situ as it passes through the ion-gated nanochannel membrane of the reactor.

In accordance with an embodiment, using the ion-gated nanochannel catalytic membrane reactor, production of dimethyl carbonate ((CH3O)2CO) may be performed by providing, in a reaction unit, methanol (CH3OH) and urea (CO(NH2)2) as reactants in a ration exceeding 8:1, for producing dimethyl carbonate ((CH3O)2CO) and ammonia (NH3) as permeates due to activation by a catalyst. Subsequently, from a separation unit, the ammonia (NH3) produced as permeate may be separated in-situ as it passes through the ion-gated nanochannel membrane of the reactor.

The proposed invention is to provide ion-gated nanochannel catalytic membrane reactors or modules or devices for chemical and fuel production which is thermodynamically limited by one of the products. More specifically, our invented ion-gated nanochannel catalytic membrane reactors or modules or devices can effectively in-situ remove small polar molecules produced in these thermodynamically limited catalytic reactions in chemical and fuel production, thereby improving the reaction efficiency.

Hereinafter, the present invention is described more specifically.

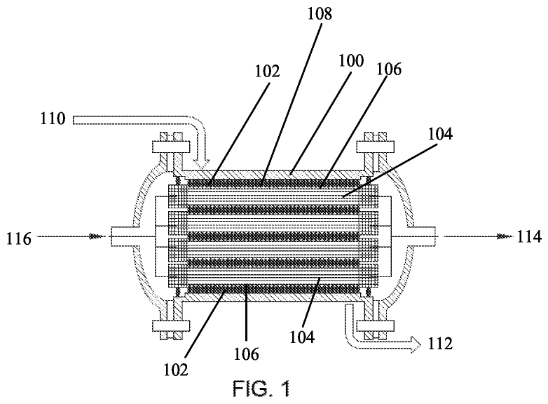

FIG. 1 depicts a schematic diagram of ion-gated nanochannel catalytic membrane reactor 100, in accordance with an embodiment of the invention. With reference to FIG. 1, there is shown an ion-gated nanochannel catalytic membrane reactor 100 having a reaction unit 102 and a separation unit 104 combined in one structure. In an embodiment, a pair of the reaction unit 102 and the separation unit 104 may form a module. The reactor 100 may include a plurality of such modules integrated within the structure of the reactor 100.

The reaction unit 102 and the separation unit 104 may be separated by an ion-gated nanochannel membrane 106. The reaction unit 102 corresponds to be section of the reactor 100 having a catalyst 108 and a plurality of reactants 110. The catalyst 108 and the plurality of reactants 110 may react in the proximity of the ion-gated nanochannel membrane 106 that may be supported on a hollow fiber support. The ion-gated nanochannel membrane 106 facilitates passing through of one or more permeates formed as a result of reaction between the plurality of reactants 110. Such permeates may be separated from the reactor 100, in-situ.

In an embodiment, the ion-gated nanochannel membrane 106 may be coated on a ceramic hollow fiber support. This enables the operation of the membrane reactor at high temperature (>300° C.) and pressure (>30 bar).

Further, the ion-gated nanochannel membrane 106 includes metal ions that are one or two of Lithium (Li), Sodium (Na), Potassium (K), Rubidium (Rb), or Cesium (Cs). The metal ions may be incorporated in the ion-gated nanochannel membrane 106 by an ion exchange mechanism with sodium zeolite membrane.

The reactor 100 may further include an first collection unit 112 and a second collection unit 114. The first collection unit 112 may allow for collection of one or more permeates due to the activation of the plurality of reactants 110 by the catalyst 108. In an embodiment, the one or more permeates collected from the first collection unit 112 may include one or more polar small molecules of the compounds formed as a result of reaction between the plurality of reactants 110, when activated. Such one or more permeates that include polar small molecules created as a result of the reaction may pass through the ion-gated nanochannel membrane 106. In an embodiment, the passing through of the polar permeates may be based on manipulation of the size of a plurality of pores of the ion-gated nanochannel membrane 106 by selecting the metal ions in accordance with the size of the polar permeates. Such one or more permeates includes the products of the reaction and continued in-situ separation of the polar small molecules results in shifting of thermodynamic equilibrium towards the continuous product formation, resulting in better yield.

Further, the one or more other permeates that include less polar large molecules created as a result of the reaction and sweep gases 116 if it is used during aforesaid chemical reaction may be collected from the second collection unit 114, if and when required. Such permeates includes the molecules of the plurality of reactants 110.

A person of ordinary skill in the art will appreciate that the size and polarity of the permeates described above are relative to the size and polarity of the molecules of the plurality of reactants 110.

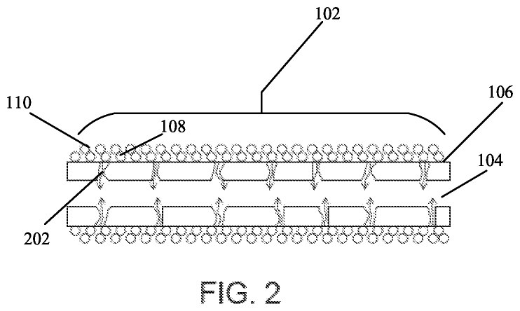

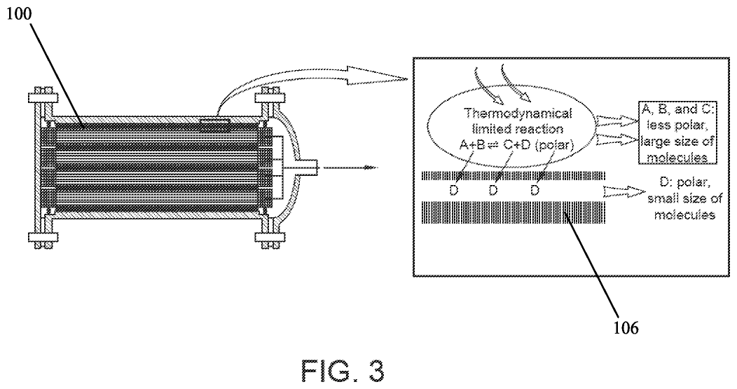

FIG. 2 shows a simplified schematic view 200 of reaction unit 102 and the separation unit 104, in accordance with an embodiment of the invention. The view 200 includes the catalyst 108, the ion-gated nanochannel membrane 106, and a porous support 202. In an embodiment, the porous support 202 may be a hollow fiber support. Element of FIG. 3 have been explained in conjunction with the elements depicted in FIG. 1.

The plurality of reactants 110 may react in the presence of the catalyst 108. The one or more permeates formed as a result of the reaction may collected using the plurality of collection units (explained in FIG. 1). The one or more permeates that include polar small molecules created as a result of the reaction may pass through the ion-gated nanochannel membrane 106. As explained in FIG. 1, a single reactor 100 may include a plurality of pair of reaction unit 102 and the separation unit 104.

FIG. 3 depicts activation of the plurality of reactants within an ion-gated nanochannel catalytic membrane reactor 100, for formation of one or more permeates, in accordance with an embodiment of the invention. Element of FIG. 3 have been explained in conjunction with the elements depicted in FIGS. 1 and 2.

With reference to FIG. 3, there is shown the reactor 100 in which the catalyst 108 activates the plurality of reactants 110 (such as A and B as shown) at the desired temperature (150-400° C.) and pressure (1-100 bar). The activation results in conversion of the plurality of reactants 110 into one or more permeates (such as A and B as shown). The one or more permeates formed may include molecules that are more polar and smaller (such as molecule D) and molecules that are less polar and larger (such as molecule C), compared to the molecules of the plurality of reactants 110 (molecules A and B) or molecule D.

The ion-gated nanochannel membrane 106, which is a ion-gated and highly selective and permeable membrane may separate the polar, small molecules by allowing such molecules to pass through the membrane. Such molecules may be collected, in-situ, from the first collection unit 112. In an embodiment, the molecules may include, either main product or by-product, such as water, ammonia. The in-situ separation of such molecules shifts the thermodynamic equilibrium towards continuous product formation (i.e., formation of molecules C and D).

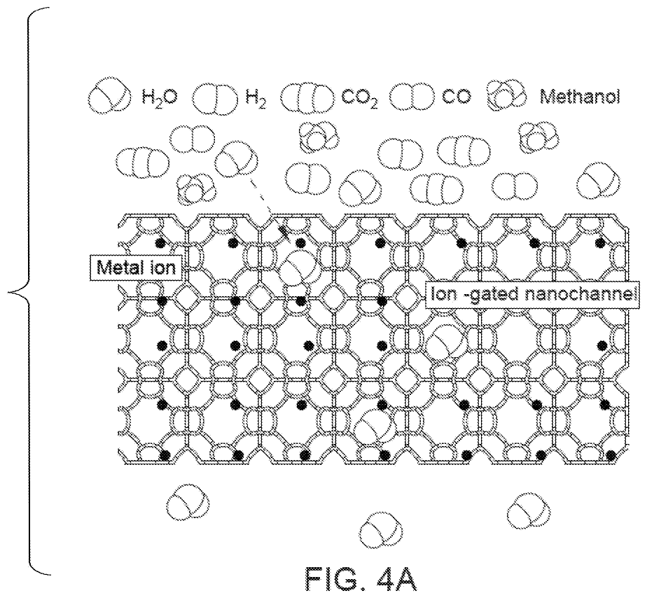

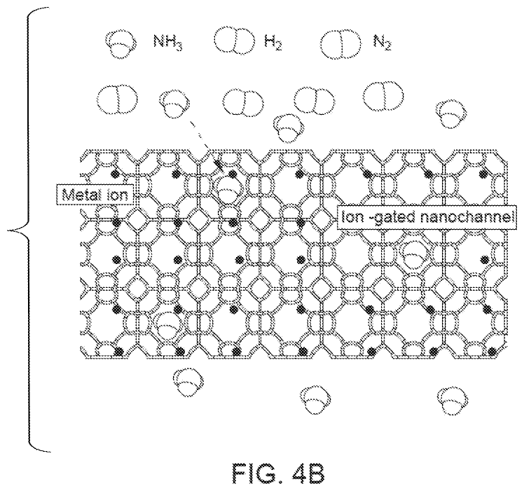

FIGS. 4A and 4B depict the separation of polar, small molecules of H2O (FIG. 4A) and NH3 (FIG. 4B) with other less polar, large molecules using the ion-gated membrane.

FIG. 4A is a schematic view that depicts the presence of metal ions in the channel of the ion-gated nanochannel membrane 106 and the permeation of polar small molecules through the ion-gated nanochannel membrane 106. Elements of FIG. 4A have been explained in conjunction with the elements of FIGS. 1 to 3.

FIG. 4A depicts separation of polar water (H2O) during hydrogenation of carbon dioxide (CO2) to methanol (CH3OH). Typically, CO2 hydrogenation to methanol is limited by thermodynamics according to the reaction below:

CO2+3H2 [symbol] CH3OH+H2O, ΔH0=−49.5 kJ/mol.

The by-product of H2O is a polar molecule with kinetic diameter of 0.265 nm, while H2 (kinetic diameter: 0.289 nm) and CO2 (kinetic diameter: 0.33 nm) are less polar molecules with larger size. In the proposed ion-gated nanochannel catalytic membrane reactor 100, the methanol (CH3OH) synthesis is activated by the catalyst 108 at moderate temperatures (150-300° C.) and pressures (10-70 bar). At the aforesaid temperature and pressure, the CO2 and H2 convert to methanol (CH3OH) and H2O. Using the ion-gated nanochannel membrane 106, the by-product H2O permeates through ion-gated nanochannel membrane and may be separated from methanol (CH3OH), CO2, and H2 in-situ, shifting the thermodynamic equilibrium towards continuous methanol (CH3OH) production. The ion-gated nanochannel membrane 106 has high selectivity of H2O over methanol (CH3OH), CO2, and H2, and enables high (>94 vol. %) purity of methanol (CH3OH) in retention side (first collection unit 112), eliminating the need for further purification.

In another example, the ion-gated nanochannel membrane 106 may be used during CO2 hydrogenation to ethanol (CH3CH2OH). The reaction of CO2 hydrogenation to ethanol (CH3CH2OH) is also reversable and thermaldynamic limited reaction:

2CO2+6H2 [symbol] CH3CH2OH+3H2O ΔH0=−86.7 kJ/mol

The by-product of H2O may be removed, in-situ, from the reaction mixture to shift towards the continuous ethanol (CH3CH2OH) production. In the proposed ion-gated nanochannel catalytic membrane reactor 100, the ethanol (CH3CH2OH) synthesis is activated by the catalyst 108 at temperatures (150-400° C.) and pressures (10-70 bar). At the aforesaid temperature and pressure, the CO2 and H2 convert to ethanol (CH3CH2OH) and H2O. Using the ion-gated nanochannel membrane 106, the by-product H2O permeates through ion-gated nanochannel membrane and may be separated from ethanol (CH3CH2OH), CO2, and H2 in-situ, shifting the thermodynamic equilibrium towards continuous olefins ethanol (CH3CH2OH) production. This results in achieving high CO2 conversion and ethanol (CH3CH2OH) yield. In addition, the ion-gated nanochannel membrane 106 has high selectivity of H2O over ethanol (CH3CH2OH), CO2, and H2, and enables high (>95 vol. %) purity of ethanol (CH3CH2OH) in retention side (first collection unit 112), eliminating the need for further purification.

In another example, the ion-gated nanochannel membrane 106 may be used during CO2 hydrogenation to olefins (CnH2n). The reaction of CO2 hydrogenation to olefins (CnH2n) is also reversable and thermaldynamic limited reaction:

nCO2+3nH2 [symbol] (-CH2-)n+2nH2O

The by-product of H2O may be removed, in-situ, from the reaction mixture to shift towards the continuous olefins (CnH2n) production. In the proposed ion-gated nanochannel catalytic membrane reactor 100, the olefins (CnH2n) synthesis is activated by the catalyst 108 at temperatures (150-400° C.) and pressures (10-70 bar). At the aforesaid temperature and pressure, the CO2 and H2 convert to olefins (CnH2n) and H2O. Using the ion-gated nanochannel membrane 106, the by-product H2O permeates through ion-gated nanochannel membrane and may be separated from olefins (CnH2n), CO2, and H2 in-situ, shifting the thermodynamic equilibrium towards continuous olefins (CnH2n) production. This results in achieving high CO2 conversion and olefins (CnH2n) yield. In addition,
the ion-gated nanochannel membrane 106 has high selectivity of H2O over olefins (CnH2n), CO2, and H2, which results olefins (CnH2n)-rich product gas in retention side (first collection unit 112).

Similarly, gasoline (C6-C10) and jet fuel range (C8-C15) hydrocarbons synthesis from CO2 is reversable and thermodynamically limited reactions and generates byproduct of H2O.

Here also, using the ion-gated membrane 106, the polar H2O may be removed from the reactor 100, driving the reaction to form gasoline (C6-C10) and jet fuel range (C8-C15) hydrocarbons. This results in achieving high CO2 conversion and hydrocarbons yield. The ion-gated nanochannel membrane 106 has high selectivity of H2O over hydrocarbons, CO2, and H2, which results gasoline (C6-C10) and jet fuel range (C8-C15)-rich product gas in retention side (first collection unit 112).

In another example, the ion-gated nanochannel membrane 106 may be used during CO2 hydrogenation to methane (CH4). The reaction of CO2 hydrogenation to methane (CH4) is also called CO2 methanation via a Sabatier Reaction.

CO2+4H2 [symbol] CH4+2H2O, ΔH0=−165 kJ/mol.

The aforesaid reaction is thermodynamically limited. The by-product of H2O may be removed, in-situ, from the reaction mixture to shift towards the continuous methane (CH4) production. In the proposed reactor 100, the CO2 methanation can be performed at moderate temperatures (150-400° C.) and pressures (1-70 bar) under CO2 methanation catalyst. This converts CO2 and H2 to methane (CH4) and H2O. By-product H2O permeates through ion-gated nanochannel membrane 106 and may be separated from methane (CH4), CO2, and H2 in-situ, shifting the thermodynamic equilibrium towards continuous methane (CH4) production. This results in achieving high CO2 conversion and methane (CH4) yield. The ion-gated nanochannel membrane 106 has high selectivity of H2O over methane, CO2, and H2, which results methane (CH4)-rich product gas in retention side (first collection unit 112). The methane (CH4)-rich product gas may easily meet the industry requirement such as gas grid feed requirement and can be directly used without the need for further purification.

FIG. 4B depicts separation ammonia (NH3) using the ion-gated nanochannel membrane during ammonia synthesis. The conversion of N2 and H2 to NH3 is limited by thermodynamics as per the following reaction:

N2+3H2 [symbol] N2NH3, ΔH0=−91.8 kJ/mol.

The product of NH3 is a polar molecule with kinetic diameter of 0.26 nm, while H2 (kineticdiameter: 0.289 nm) and N2 (kinetic diameter: 0.364 nm) are less polar molecules with larger size. In the proposed ion-gated nanochannel catalytic membrane reactor 100, the ammonia synthesis catalyst activates N2 and H2 at moderate temperatures (300-400° C.) and pressures (10-70 bar) and converts them to NH3. Synthesized ammonia (NH3) permeates through ion-gated nanochannel membrane 106 and is separated from N2 and H2 in-situ from the first collection unit 112. This shifts the thermodynamic equilibrium towards continuous ammonia (NH3) production. The ion-gated nanochannel membrane 106 has high selectivity of ammonia (NH3) over N2 and H2, which enables high (˜99 vol. %) purity of ammonia (NH3) in permeate side, eliminating the need for further purification.

In another example, the ion-gated nanochannnel catalytic membrane reactor 100 may be used for dimethyl carbonate (DMC) synthesis from methanol and urea. DMC can be synthesized from methanol and urea by two reactions:

CH3OH+CO(NH2)2 [symbol] CH3OCONH2+NH3; and

CH3OH+CH3OCONH2 [symbol] (CH3O)2CO+NH3

The second reaction is strongly limited by the thermodynamic equilibrium due to the accumulation of polar, byproduct ammonia (NH3) in the reaction system, leading to a low DMC yield. In addition, high ratio of methanol to urea (molar ratio >8:1) in feedstock, beneficial for driving reaction forward and improving DMC selectivity, requires an energy-intensive and non-economic complex process for DMC purification. Solving the problem identified above, the proposed ion-gated nanochannel catalytic membrane reactor 100 separate ammonia (NH3) in-situ, shifting the thermodynamic equilibrium towards DMC formation. The removal of ammonia (NH3) is also expected to alleviate side reactions and improve the selectivity of DMC. The separated ammonia (NH3) may be recycled and react with CO2, captured from various sources, to produce urea via commercially available process.

The proposed ion-gated nanochannel catalytic membrane reactor 100, by combining highly selective ion-gated nanochannel membrane 106 with catalyst in one-unit, allows reaction and separation of polar, small product in-situ, thereby overcoming the thermodynamic and kinetic barriers and achieving high reactant conversion and product yield. The advantages of our invention include double or triple reactant conversion and production yield compared to traditional fixed bed reactor. Further, the reactor 100 leads to direct production of high purity (>95%) desired product. The proposed reactor 100 extends the catalyst lifetime due to the removal of polar molecules in situ. Furthermore, the proposed reactor 100 eliminates energy-intensive separation steps such as distillation or cryogenic condensation process. The proposed reactor 100 simplifies the process by combining the reaction and separation in one unit. Also, the proposed reactor 100 decreases (>50%) energy consumption and reduces (>50%) operating cost, and lowers overall production cost compared to the traditional fixed bed reactors.

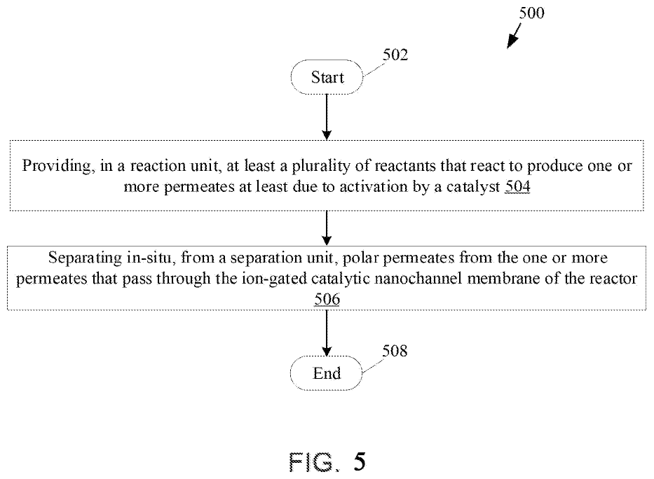

FIG. 5 depicts a flow diagram of a method for production of compounds using an ion-gated nanochannel catalytic membrane reactor, in accordance with some embodiments of the present disclosure. With reference to FIG. 5, there is shown a flow chart 500. The flow chart 500 is described in conjunction with FIGS. 1 and 4B. The process starts at step 502 and proceeds to step 504.

At step 504, in the reaction unit 102, at least a plurality of reactants that react to produce one or more permeates at least due to activation by a catalyst may be provided. At step 506, from a separation unit 104, polar permeates from the one or more permeates that pass through the ion-gated nanochannel membrane of the reactor may be separated, in situ. The separation unit 104 may include the ion-gated nanochannel membrane 106 supported on a hollow fiber support. The control passes to end step 508.

In the drawings and specification, there have been disclosed exemplary embodiments of the present disclosure. Although specific terms are employed, they are used in a generic and descriptive sense only and not for purposes of limitation, the scope of the present disclosure being defined by the following claims. Those skilled in the art will recognize that the present disclosure admits of a number of modifications, within the spirit and scope of the inventive concepts, and that it may be applied in numerous applications, only some of which have been described herein. It is intended by the following claims to claim all such modifications and variations, which fall within the true scope of the present disclosure.

I/We claim:

1. An ion-gated nanochannel catalytic membrane reactor, the reactor comprising:
a reaction unit comprising at least a plurality of reactants that react to produce one or more permeates at least due to activation by a catalyst, and
a separation unit comprising an ion-gated nanochannel membrane supported on a hollow fiber support, wherein polar permeates from the one or more permeates pass through the ion-gated nanochannel membrane and are separated in-situ.

2. The ion-gated nanochannel catalytic membrane reactor of claim 1, wherein the ion-gated nanochannel membrane is coated on a ceramic hollow fiber support.

3. The ion-gated nanochannel catalytic membrane reactor of claim 1, wherein the ion-gated nanochannel membrane comprises metal ions that are one or two of Lithium (Li), Sodium (Na), Potassium (K), Rubidium (Rb), or Cesium (Cs).

4. The ion-gated nanochannel catalytic membrane reactor of claim 3, wherein the metal ions are incorporated in the ion-gated nanochannel membrane by an ion exchange mechanism with sodium zeolite membrane.

5. The ion-gated nanochannel catalytic membrane reactor of claim 4, wherein the passing through of the polar permeates, is based on manipulation of the size of a plurality of pores of the ion-gated nanochannel membrane by selecting the metal ions in accordance with the size of the polar permeates.

6. The ion-gated nanochannel catalytic membrane reactor of claim 1, wherein production of methanol (CH₃OH) is performed using the ion-gated nanochannel catalytic membrane reactor by:
providing, in a reaction unit, carbon dioxide (CO₂) and hydrogen (H₂) as reactants for producing methanol (CH₃OH) and polar water (H₂O) as permeates due to activation by a catalyst, an ambient temperature (150° C.-300° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

7. The ion-gated nanochannel catalytic membrane reactor of claim 1, wherein production of ethanol (CH₃CH₂OH) is performed using the ion-gated nanochannel catalytic membrane reactor by:
providing, in a reaction unit, carbon dioxide (CO₂) and hydrogen (H₂) as reactants for producing ethanol (CH₃CH₂OH) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

8. The ion-gated nanochannel catalytic membrane reactor of claim 1, wherein production of olefins (CₙH₂ₙ) is performed using the ion-gated nanochannel catalytic membrane reactor by:
providing, in a reaction unit, carbon dioxide (CO₂) and hydrogen (H₂) as reactants for producing olefins (CₙH₂ₙ) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

9. The ion-gated nanochannel catalytic membrane reactor of claim 1, wherein production of gasoline (C6-C10) and jet fuel range (C8-C15) hydrocarbons is performed using the ion-gated nanochannel catalytic membrane reactor by:
providing, in a reaction unit, carbon dioxide (CO₂) and hydrogen (H₂) as reactants for producing gasoline (C6-C10) and jet fuel range (C8-C15) hydrocarbons and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

10. The ion-gated nanochannel catalytic membrane reactor of claim 1, wherein production of methane (CH₄) is performed using the ion-gated nanochannel catalytic membrane reactor by:
providing, in a reaction unit, carbon dioxide (CO₂) and hydrogen (H₂) as reactants for producing methane (CH₄) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

11. The ion-gated nanochannel catalytic membrane reactor of claim 1, wherein production of ammonia (NH₃) is performed using the ion-gated nanochannel catalytic membrane reactor by:
providing, in a reaction unit, nitrogen (N2) and hydrogen (H₂) as reactants for producing ammonia (NH₃) as permeate due to activation by a catalyst, an ambient temperature (300° C.-400° C.), and pressure of (10-70 bar);
separating in-situ, from a separation unit, the ammonia (NH₃) produced as permeate as it passes through the ion-gated nanochannel membrane of the reactor, wherein the separation unit comprises an ion-gated nanochannel membrane supported on a hollow fiber support.

12. The ion-gated nanochannel catalytic membrane reactor of claim 1, wherein production of dimethyl carbonate ((CH₃O)₂CO) is performed using the ion-gated nanochannel catalytic membrane reactor by:
providing, in a reaction unit, methanol (CH₃OH) and urea (CO(NH₂)₂) as reactants in a ration exceeding 8:1, for producing dimethyl carbonate ((CH₃O)₂CO) and ammonia (NH₃) as permeates due to activation by a catalyst;
separating in-situ, from a separation unit, the ammonia (NH₃) produced as permeate as it passes through the ion-gated nanochannel membrane of the reactor, wherein the separation unit comprises an ion-gated nanochannel membrane supported on a hollow fiber support.

13. A method for production of compounds using an ion-gated nanochannel catalytic membrane reactor, the method comprising:
providing, in a reaction unit, at least a plurality of reactants that react to produce one or more permeates at least due to activation by a catalyst;
separating in-situ, from a separation unit, polar permeates from the one or more permeates that pass through the ion-gated nanochannel membrane of the reactor, wherein the separation unit comprises an ion-gated nanochannel membrane supported on a hollow fiber support.

14. The method of claim 13, wherein the ion-gated nanochannel membrane is coated on a ceramic hollow fiber support.

15. The method of claim 13, wherein the ion-gated nanochannel membrane comprises metal ions that are one or two of Lithium (Li), Sodium (Na), Potassium (K), Rubidium (Rb), or Cesium (Cs).

16. The method of claim 15, wherein the metal ions are incorporated in the ion-gated nanochannel membrane by an ion exchange mechanism with sodium zeolite membrane.

17. The method of claim 15, wherein the passing through of the polar permeates, is based on manipulation of the size of a plurality of pores of the ion-gated nanochannel membrane by selecting the metal ions in accordance with the size of the polar permeates.

18. The method of claim 13, wherein production of methanol (CH₃OH) is performed using the ion-gated nanochannel catalytic membrane reactor by:
providing, in a reaction unit, carbon dioxide (CO2) and hydrogen (H₂) as reactants for producing methanol (CH₃OH) and polar water (H₂O) as permeates due to activation by a catalyst, an ambient temperature (150° C.-300° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

19. The method of claim 13, wherein production of ethanol (CH₃CH₂OH) is performed using the ion-gated nanochannel catalytic membrane reactor by:
providing, in a reaction unit, carbon dioxide (CO2) and hydrogen (H₂) as reactants for producing ethanol (CH₃CH₂OH) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

20. The method of claim 13, wherein production of olefins (CₙH₂ₙ) is performed using the ion-gated nanochannel catalytic membrane reactor by:
providing, in a reaction unit, carbon dioxide (CO₂) and hydrogen (H₂) as reactants for producing olefins (CₙH₂ₙ) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

21. The method of claim 13, wherein production of gasoline (C6-C10) and jet fuel range (C8-C15) hydrocarbons is performed using the ion-gated nanochannel catalytic membrane reactor by:
providing, in a reaction unit, carbon dioxide (CO₂) and hydrogen (H₂) as reactants for producing gasoline (C6-C10) and jet fuel range (C8-C15) hydrocarbons and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

22. The method of claim 13, wherein production of methane (CH₄) is performed using the ion-gated nanochannel catalytic membrane reactor by:
providing, in a reaction unit, carbon dioxide (CO₂) and hydrogen (H₂) as reactants for producing methane (CH₄) and polar water (H₂O) as permeates due to activation by a catalyst, an ambient temperature (150° C.-400° C.), and pressure of (10-70 bar);
separating in-situ, from a separation unit, the polar water (H₂O) produced as permeate as it passes through the ion-gated nanochannel membrane of the reactor, wherein the separation unit comprises an ion-gated nanochannel membrane supported on a hollow fiber support.

23. The method of claim 13, wherein production of ammonia (NH₃) is performed using the ion-gated nanochannel catalytic membrane reactor by:
providing, in a reaction unit, nitrogen (N2) and hydrogen (H₂) as reactants for producing ammonia (NH₃) as permeate due to activation by a catalyst, an ambient temperature (300° C.-400° C.), and pressure of (10-70 bar);
separating in-situ, from a separation unit, the ammonia (NH₃) produced as permeate as it passes through the ion-gated nanochannel membrane of the reactor, wherein the separation unit comprises an ion-gated nanochannel membrane supported on a hollow fiber support.

24. The method of claim 13, wherein production of dimethyl carbonate ((CH₃O)₂CO) is performed using the ion-gated nanochannel catalytic membrane reactor by:
providing, in a reaction unit, methanol (CH₃OH) and urea (CO(NH₂)₂) as reactants in a ration exceeding 8:1, for producing dimethyl carbonate ((CH₃O)₂CO) and ammonia (NH₃) as permeates due to activation by a catalyst;
separating in-situ, from a separation unit, the ammonia (NH₃) produced as permeate as it passes through the ion-gated nanochannel membrane of the reactor, wherein the separation unit comprises an ion-gated nanochannel membrane supported on a hollow fiber support.
